(19) United States
(12) Patent Application Publication
(10) Pub. No.: US 2014/0128647 A1
(43) Pub. Date: May 8, 2014

(54) METHOD AND APPARATUS FOR REDUCING AN AROMATIC CONCENTRATION IN A HYDROCARBON STREAM

(71) Applicant: UOP LLC, Des Plaines, IL (US)

(72) Inventors: David James Shecterle, Arlington Heights, IL (US); Jayant K. Gorawara, Buffalo Grove, IL (US); Henry Rastelli, Gurnee, IL (US)

(73) Assignee: UOP LLC, Des Plaines, IL (US)

(21) Appl. No.: 13/669,816

(22) Filed: Nov. 6, 2012

(51) Int. Cl. C07C 7/163; C07C 5/13; C07C 7/12; B01J 10/00

(52) U.S. Cl. 585/253; 422/187; 585/258; 585/802

(57) ABSTRACT

Methods and apparatuses for reducing an aromatic concentration in a hydrocarbon stream are provided. In an embodiment, a method for reducing an aromatic concentration in a hydrocarbon stream includes saturating aromatics in the hydrocarbon stream to form a low aromatic hydrocarbon stream comprising no more than about 2 weight percent (wt %) aromatics. Further, the method includes passing the low aromatic hydrocarbon stream through an adsorption zone to remove aromatics therefrom to form an aromatic-depleted product stream comprising less than about 10 weight parts per million (wppm) aromatics.

### TECHNICAL FIELD

The technical field generally relates to methods and apparatuses for reducing aromatic concentrations in hydrocarbon streams, and more particularly relates to methods and apparatuses for forming aromatic-depleted product streams.

### BACKGROUND

Commercial grade hexane is a high value product used as a solvent in the food and energy industries. During typical processing of naphtha to form commercial grade hexane, aromatics are separated and removed. Aromatics may be undesirable for performance or environmental reasons. For example, benzene is a known carcinogen and must be reduced to very low levels in many solvents and chemical products.

The commercial grade product specification for normal hexane is less than 10 weight parts per million (wppm) benzene. For food grade hexane, the specification requires less than 3 wppm benzene. For hydrocarbon streams such as those processed to form normal hexane product, it is economically difficult to reduce benzene concentrations to the specified levels through fractionation because the relative volatility of benzene is very close to the relative volatilities of other stream components.

In addition to normal hexane, it may be desirable to remove aromatics from other products, such as cyclohexane, that are formed through the processing of hydrocarbon streams. For example, high purity cyclohexane is often formed by the hydrogenation of high purity benzene. It is important to achieve very low concentrations of benzene in the high purity cyclohexane product. In a typical process for forming high purity cyclohexane, multi-bed reactor systems are utilized to remove aromatics to achieve the required low concentrations of benzene. However, operation of these systems is often expensive as they typically run at high pressure.

Accordingly, it is desirable to provide novel methods and apparatuses for reducing aromatic concentrations in hydrocarbon streams. It is also desirable to provide methods and apparatuses for forming a benzene-depleted C6 product stream. Also, it is desirable to provide such methods and apparatuses that operate economically. Furthermore, other desirable features and characteristics will become apparent from the subsequent detailed description and the appended claims, taken in conjunction with the accompanying drawings and the foregoing technical field and background.

### BRIEF SUMMARY

Methods and apparatuses for reducing an aromatic concentration in a hydrocarbon stream are provided. In one embodiment, a method for reducing an aromatic concentration in a hydrocarbon stream includes saturating aromatics in the hydrocarbon stream to form a low aromatic hydrocarbon stream comprising no more than about 2 weight percent (wt %) aromatics. Further, the method includes passing the low aromatic hydrocarbon stream through an adsorption zone to remove aromatics therefrom to form an aromatic-depleted product stream comprising less than about 10 weight parts per million (wppm) aromatics.

In another embodiment, a method for forming a benzene-depleted C6 product stream is provided. The method for forming a benzene-depleted C6 product stream includes fractionating a hydrocarbon stream to form a C6-concentrated stream comprising C6 paraffins, C6 olefins, C6 naphthenes, and no more than about 2 weight percent (wt %) benzene. Further, the method includes adsorbing benzene from the C6-concentrated stream to form the benzene-depleted C6 product stream comprising less than about 10 weight parts per million (wppm) benzene.

In another embodiment, an apparatus for reducing an aromatic concentration in a hydrocarbon stream is provided. The apparatus for reducing an aromatic concentration in a hydrocarbon stream includes a saturation zone configured to receive the hydrocarbon stream and to saturate aromatics therein to form a low aromatic hydrocarbon stream comprising no more than about 2 weight percent (wt %) aromatics. Also, the apparatus includes an adsorption zone configured to receive the low aromatic hydrocarbon stream from the saturation zone and to remove aromatics therefrom to form an aromatic-depleted product stream comprising less than about 10 weight parts per million (wppm) aromatics.

### BRIEF DESCRIPTION OF THE DRAWINGS

Embodiments of methods and apparatuses for reducing aromatic concentrations in hydrocarbon streams will hereinafter be described in conjunction with the following drawing figures, wherein like numerals denote like elements, and wherein:

FIG. 1 is a schematic diagram of an embodiment of an apparatus and method for reducing aromatic concentrations in hydrocarbon streams including a processing zone, a fractionation zone, and an adsorption zone in accordance with an embodiment;

FIG. 2 is a schematic diagram of an embodiment of the processing zone of FIG. 1;

FIG. 3 is a schematic diagram of another embodiment of the processing zone of FIG. 1;

FIG. 4 is a schematic diagram of an embodiment of the fractionation zone of FIG. 1;

FIG. 5 is a schematic diagram of an embodiment of the adsorption zone of FIG. 1;

FIG. 6 is a schematic diagram of another embodiment of the adsorption zone of FIG. 1; and

FIG. 7 is a schematic diagram of another embodiment of the adsorption zone of FIG. 1.

### DETAILED DESCRIPTION

The following detailed description is merely exemplary in nature and is not intended to limit the methods or apparatuses for reducing aromatic concentrations in hydrocarbon streams. Furthermore, there is no intention to be bound by any theory presented in the preceding background or the following detailed description.

Methods and apparatuses for reducing aromatic concentrations in hydrocarbon streams are provided herein. The methods and apparatuses enable hydrocarbon product streams to be obtained with low levels of aromatics, such as no more than about 10 wppm aromatics. Such low levels of aromatics are possible because the hydrocarbon stream is first processed to saturate the aromatics therein and is then passed through an adsorption zone where remaining unconverted aromatics are adsorbed.

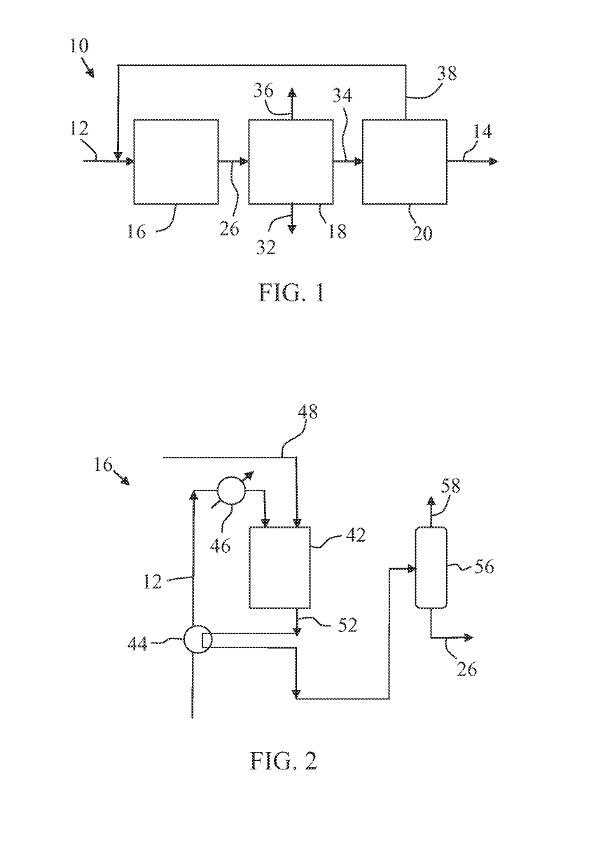

In an embodiment, and as shown in FIG. 1, an apparatus 10 for reducing aromatic concentrations in hydrocarbon streams receives and processes a hydrocarbon stream 12 or feedstock to form an aromatic-depleted product stream 14. The apparatus 10 includes a processing zone 16, a fractionation zone 18, and an adsorption zone 20. As shown, the processing zone 16 receives the hydrocarbon stream 12. Suitable hydrocarbon feedstocks include those having hydrocarbon fractions that include unbranched C₄ to C₇ hydrocarbons, i.e., normal and cyclic paraffins, or those comprising high purity benzene. In an embodiment, the hydrocarbon stream 12 is composed of at least 50 wt % of hydrocarbons with five or six carbon atoms. In another embodiment, the hydrocarbon stream 12 is rich in unbranched C₄ to C₇ hydrocarbons, meaning that the hydrocarbon stream 12 has at least 10 wt % of unbranched C₄ to C₇ hydrocarbons. Examples of suitable hydrocarbon feedstocks include hydrocarbon streams having a majority of paraffins with from four to six carbon atoms, with only residual amounts of other hydrocarbons present, a mixture of such hydrocarbon streams, or streams of substantially pure aromatics, such as benzene. As used herein, “residual” refers to amounts that are at or below separation thresholds for the process referred to, and are typically amounts of less than or equal to about 1 wt % based upon the reference composition. Other useful hydrocarbon feedstocks include natural gasoline, straight run naphtha, gas oil condensate, raffinates, reformate, field butanes, and straight run distillates having distillation end points of about 77° C. In other embodiments, the hydrocarbon stream 12 may also contain low concentrations of unsaturated hydrocarbons, hydrocarbons having more than seven carbon atoms, and cyclic hydrocarbons.

As discussed below, the hydrocarbon stream 12 is processed in the processing zone 16 in order to form a desired processed stream 26. In exemplary embodiments, double bonds in aromatic molecules in the hydrocarbon stream 12 are saturated during processing, such that the processed stream 26 includes decreased amounts of aromatics. In the exemplary embodiment of FIG. 1, the processed stream 26 exits the processing zone 16 and is received by the fractionation zone 18. As shown, the fractionation zone 18 separates the processed stream 26 into two or more cuts or fractions 32, 34, and 36. One of the fractions 32, 34 and 36 will comprise a low aromatic hydrocarbon stream. As used herein, “low aromatic” refers to a stream comprising no more than about 2 weight percent (wt %). In certain embodiments, the low aromatic hydrocarbon stream will be concentrated in normal hexane, i.e., it will contain more than 20 wt % normal hexane, such as more than 40 wt % normal hexane. In FIG. 1, the low aromatic hydrocarbon stream is formed as fraction 34 and is delivered to the adsorption zone 20 from the fractionation zone 18.

In the adsorption zone 20, aromatics are removed from the low aromatic hydrocarbon stream in fraction 34. As a result, the aromatic-depleted product stream 14 is formed and exits the adsorption zone 20. Also, a stream 38 of desorbed aromatics may be removed from the adsorption zone 20 and recycled to the processing zone 16 as discussed below. In an exemplary embodiment, the adsorption zone 20 removes a sufficient amount of aromatics from the low aromatic hydrocarbon stream in fraction 34 to provide the aromatic-depleted product stream 14 with an aromatic concentration of less than 10 weight parts per million (wppm). Further, in an embodiment, the adsorption zone 20 removes a sufficient amount of aromatics from the low aromatic hydrocarbon stream in fraction 34 to provide the aromatic-depleted product stream 14 with an aromatic concentration of less than about 3 wppm.

Referring now to FIG. 2, an embodiment of the processing zone 16 is illustrated. In FIG. 2, the processing zone 16 is a saturation zone, and the double bonds (or aromatic bonds) in aromatics in the hydrocarbon stream 12 are saturated with hydrogen during processing. As shown, the processing zone 16 includes a saturation reactor 42 which holds a fixed bed of catalyst for promoting the saturation/hydrogenation of benzene. Suitable hydrogenation will provide a metallic function to promote hydrogen transfer without any substantial acid function that would lead to undesirable cracking. Preferred catalyst compositions will include platinum group, tin or cobalt and molybdenum metals on suitable refractory inorganic oxide supports such as alumina. The alumina is preferably an anhydrous gamma-alumina with a high degree of purity. The term “platinum group metals” refers to noble metals excluding silver and gold which are selected from the group consisting of platinum, palladium, germanium, ruthenium, rhodium, osmium, and iridium. Such catalysts will provide satisfactory aromatic saturation at the operating conditions including temperatures of from about 250° C. to about 320° C. (about 480° F. to about 600° F.), preferably from about 260° C. to about 290° C. (about 500° F. to about 550° F.), pressures of from 2070 to 4820 kPa (300 to 700 psig), preferably from 2760 to 3450 kPa (400 psi to 500 psi). An exemplary catalyst is a noble metal catalyst that is selective and has no measureable side reactions. With the appropriate catalyst, no cracking of the hydrocarbons occurs and no coke forms on the catalyst to reduce activity.

In FIG. 2, the hydrocarbon stream 12 is heated by heat exchanger 44 and may be heated by a preheater 46 before being pumped to the saturation reactor 42. Typically, the preheater 46 is only used to heat the hydrocarbon stream 12 during start up, as the heat of reaction in the saturation reactor 42 is sufficient to provide the required heat input to the hydrocarbon stream 12 via the heat exchanger 44 when the saturation reactor 42 is on-line.

As shown in FIG. 2, hydrogen 48 is also delivered to the saturation reactor 42. While the hydrogen 48 is shown being delivered directly to the saturation reactor 42, it is contemplated that the hydrogen 48 be combined with the hydrocarbon stream 12 upstream of the saturation reactor 42. In an exemplary embodiment, a slight excess of hydrogen 48 above the stoichiometric level is provided. For benzene saturation, three moles of hydrogen are required for each mole of benzene saturated. Within the saturation reactor 42, the double bonds in benzene and other aromatics in the hydrocarbon stream 12 are saturated with hydrogen 48 at moderate process conditions. As a result of saturation of aromatics, benzene is converted to cyclohexane. As the benzene-cyclohexane equilibrium is strongly influenced by temperature and pressure, reaction conditions must be selected and monitored carefully. The saturation process is highly exothermic and the high heat of reaction associated with benzene saturation is managed to control the temperature rise across the saturation reactor 42.

An exemplary saturated effluent 52 exits the saturation reactor 42 and is heat exchanged with the hydrocarbon stream 12 at heat exchanger 44 to provide sufficient heat for the catalytic reaction in the saturation reactor 42 as described above. The saturated effluent 52 may then be delivered to a stabilizer 56. The stabilizer 56 removes lights in an overhead stream 58 and isolates the processed stream 26 as a stabilized low aromatic hydrocarbon stream comprising a low amount of benzene, such as less than about 2 wt % benzene, for example less than 1 wt % benzene, such as less than 500 wppm benzene, or less than 10 wppm benzene in an exemplary embodiment.

FIG. 3 illustrates another embodiment of the processing zone 16. In FIG. 3, the processing zone 16 is an isomerization zone, and aromatics in the hydrocarbon stream 12 are saturated with hydrogen during processing while paraffins in the hydrocarbon stream 12 are isomerized to isoparaffins. As shown, the processing zone 16 includes an isomerization apparatus 60 that receives the hydrocarbon stream 12 and hydrogen 62. The isomerization apparatus 60 includes catalysts that support the isomerization and saturation reactions. Isomerization apparatuses are known in the art and that can be employed to utilize fixed-bed systems, moving-bed systems, fluidized-bed systems, or batch-type systems. The hydrocarbon stream 12 may be in the liquid phase, a mixed liquid-vapor phase, or a vapor phase when contacted with the isomerization catalyst. Suitable isomerization apparatuses with a separator and a recycle gas compressor, without a separator and a recycle gas compressor, and without hydrogen recycle are all known in the art and are suitable for use in the methods and apparatuses 10 described herein.

Operating conditions within the isomerization apparatus 60 are selected to maximize the saturation of aromatics and/or the production of branched hydrocarbons from unbranched hydrocarbons that are introduced therein. Operating conditions within the isomerization apparatus 60 are dependent upon various factors including, but not limited to, feed severity and catalyst type, and those of skill in the art are readily able to identify appropriate operating conditions within the isomerization apparatus 60 to maximize saturation of aromatics in the hydrocarbon stream 12. In an embodiment, when chlorided alumina and sulfated zirconia isomerization catalysts are used, a temperature within the processing zone 16 may be from about 90° C. to about 225° C. In another embodiment, when zeolitic isomerization catalysts are used, a temperature within the isomerization apparatus 60 may be from about 90° C. to about 290° C. The isomerization apparatus 60 may be maintained over a wide range of pressures, such as from about 100 kPa to about 10 MPa, or from about 0.5 MPa to about 4 MPa. A feed rate of all hydrocarbons to the isomerization apparatus 60 can also vary over a wide range, such as at liquid hourly space velocities of from about 0.2 to about 25 volumes of hydrocarbon per hour per volume of isomerization catalyst, such as from about 0.5 to 15 hr⁻¹.

For embodiments utilizing chlorided alumina catalysts in the isomerization apparatus 60, the processing zone 16 can include one or more drying zones, such as a drying zone 64 and a drying zone 66. The drying zone 64 may include a first fluid drying unit 68, and the drying zone 66 may include a second fluid drying unit 70. The drying zone 64 receives the hydrocarbon stream 12, and the drying zone 66 receives the hydrogen 62.

Although not shown, it should be understood that fluid transfer devices, such as pumps and compressors, can be used to transport, respectively, the hydrocarbon stream 12 and the hydrogen 62. Alternatively, either fluid can be of sufficient pressure so as to not require such devices. While FIG. 3 illustrates mixing of the hydrocarbon stream 12 and the hydrogen 62 upstream of the isomerization apparatus 60, the hydrocarbon stream 12 and the hydrogen 62 may be combined at the isomerization apparatus 60.

The exemplary isomerization apparatus 60 includes a first reactor 74 and a second reactor 76 in series with the first reactor 74. Although only the first reactor 74 and second reactor 76 are depicted, it should be understood that the processing zone 16 can further include other equipment or vessels, such as one or more heaters, a recycle gas compressor, a separator vessel, a stabilizer, and additional reactors. Alternatively, the reactors 74 and 76 can be placed in single operation. The reactors 74 and 76 include catalysts for isomerizing the unbranched hydrocarbons and for saturating aromatics that are introduced into the isomerization apparatus 60. Isomerization of unbranched-hydrocarbons produces branched hydrocarbons which are included in the processed stream 26. Saturation of aromatics produces saturated hydrocarbons that are also included in the processed stream 26. Suitable catalysts are known in the art and can be amorphous (e.g., based upon an amorphous inorganic oxide), crystalline (e.g., based upon a crystalline inorganic oxide), or a mixture of both. Isomerization catalyst containing a crystalline inorganic oxide generally contains an amorphous matrix or binder. The crystalline inorganic oxide can be a molecular sieve or a non-molecular sieve, or a mixture of a molecular sieve and a non-molecular sieve can be used. The molecular sieve can be zeolitic or non-zeolitic, or a mixture of a zeolite and a non-zeolite can be used. The isomerization catalyst may include platinum on mordenite, aluminum chloride on alumina, and platinum on sulfated or tungstated metal oxides such as zirconia. The isomerization catalyst may include a platinum group metal such as platinum on a chlorided alumina base, such as an anhydrous gamma-alumina. A chloride component present in the isomerization catalyst, termed in the art “a combined chloride”, may be present in an amount from about 2% to about 10% by weight, such as from about 5% to about 10% by weight, based on the total weight of the isomerization catalyst.

Regardless of the design of isomerization apparatus 60, normal paraffins entering the isomerization apparatus 60 in the hydrocarbon stream 12 are rearranged or restructured into more complex molecular shapes having higher octane values during isomerization. Further, benzene is saturated with hydrogen and converted to cyclohexane. The processed stream 26 exits the isomerization apparatus 60 as an isomerization effluent containing the higher octane components, cyclohexane, and a reduced concentration of benzene, such as less than about 2 wt % benzene, for example less than about 1 wt % benzene, such as less than about 10 ppm.

In an exemplary embodiment, the hydrogen 62 is provided in an amount that provides a molar hydrogen-to-hydrocarbon ratio of less than or equal to about 0.05 in the processed stream 26 when operating without hydrogen recycle, which provides sufficient excess hydrogen 62 to ensure that any unsaturated hydrocarbons that are introduced into the processing zone 16 are properly saturated. Although no net hydrogen 62 is consumed during isomerization of hydrocarbons in the processing zone 16, the processing zone 16 has a net consumption of hydrogen 62 that is associated with cracking, disproportionation, and olefin and aromatics saturation, and the excess hydrogen 62 ensures that sufficient amounts of hydrogen 62 are present in the processing zone 16 to enable the isomerization and saturation reactions to occur.

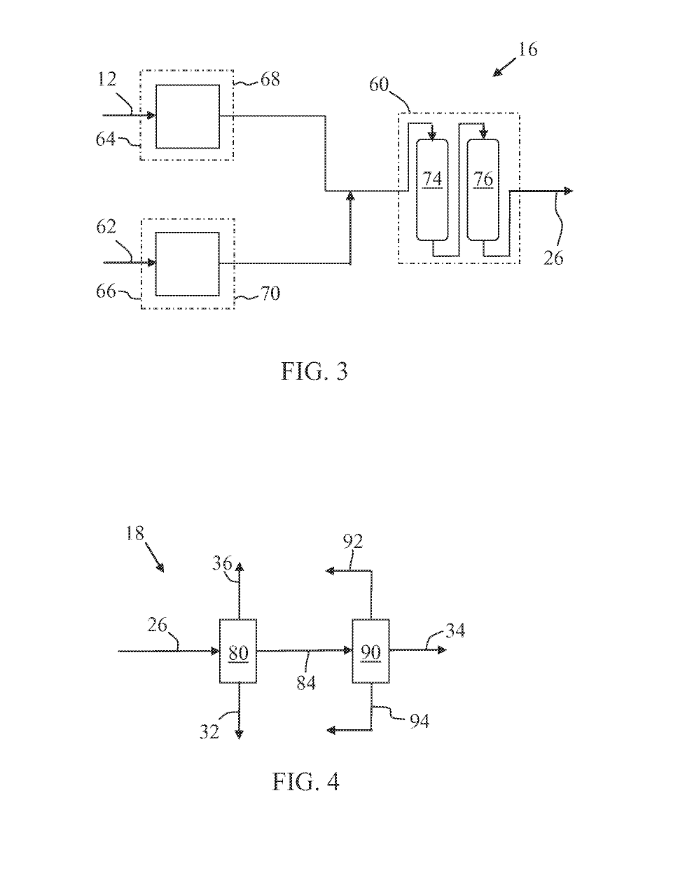

Referring now to FIG. 4, an embodiment of the fractionation zone 18 is illustrated. In FIG. 4, the processed stream 26 is separated into bottoms fraction 32 that generally includes hydrocarbons having a higher boiling point than normal and cyclic hexane and monomethyl pentanes, a low aromatic hydrocarbon fraction 34 that generally includes normal hexane and a low level of aromatics that were not saturated in the processing zone 16, and overhead fraction 36 that generally includes branched hydrocarbons. The overhead fraction 36 includes a higher content of branched hydrocarbons than the fraction 34, and it is to be appreciated that the overhead fraction 36 and the fraction 34 can include chemical species in addition to branched hydrocarbons, normal hexane and aromatics.

As indicated in FIG. 1, the processed stream 26 is separated in a fractionation zone 18 that is in fluid communication with the processing zone 16. As shown in FIG. 4, the fractionation zone 18 includes a fractionation unit 80 that receives the processed stream 26. In an embodiment, the fractionation unit 80 separates the bottoms fraction 32 and the overhead fraction 36 from the processed stream 26. An exemplary bottoms fraction 32 largely contains cyclohexane, methylcyclohexane, and methylcyclopentane, while an exemplary overhead fraction 36 largely contains butanes, isopentane, normal pentane, cyclopentane, dimethylbutanes and methylpentanes. A side draw stream 84, largely containing dimethylbutanes, methylpentanes, normal hexane, methylcyclopentane, cyclohexane and aromatics, exits the fractionation unit 80.

In FIG. 4, the side draw stream 84 exits the fractionation unit 80 and is received by a fractionation unit 90. Fractionation unit 90 separates the low aromatic hydrocarbon fraction 34 as a side draw. The low aromatic hydrocarbon fraction 34 may largely contain normal hexane, methylpentanes, and methylcyclopentane. Further, fractionation unit 90 separates a light recycle overhead stream 92 and heavy recycle bottom stream 94 which are returned to the processing zone 16 and can be mixed with the hydrocarbon stream 12 upstream of the processing zone 16. The light recycle overhead stream 92 may largely contain dimethylbutanes, methylpentanes and normal hexane, which the heavy recycle bottom stream 94 may largely contain methylcyclopentane, cyclohexane, cyclopentanes, cyclohexanes, and hexane.

In the embodiment of FIG. 4, the overhead fraction 36 includes branched hydrocarbons having six or fewer carbon atoms and linear hydrocarbons having five or fewer carbon atoms. Specifically, the overhead fraction 36 generally contains pentanes, and dimethylbutanes. The low aromatic hydrocarbon fraction 34 includes normal hexane, cyclic hydrocarbons, monomethyl-branched pentane, and a low level of aromatics. Due to inefficiencies in separation, it is to be appreciated that residual amounts of various hydrocarbons can be present in the respective fractions 34, 36, and that complete separation is rarely feasible. In an embodiment, the low aromatic hydrocarbon fraction 34 includes at least about 40 wt. % normal hexane and less than about 2 wt % aromatics, such as benzene, with a balance of the fraction 34 being predominantly methylpentanes, cyclohexane, and methylcyclopentane, and including residual amounts of dimethylbutanes and heptanes. Hydrocarbons having a higher boiling point than normal and cyclic hexane and monomethyl pentanes are withdrawn from the fractionation unit 80 in the bottoms fraction 32, although cyclic hexanes are generally present in the bottoms fraction 32 as well. Examples of hydrocarbons having a higher boiling point than normal and cyclic hexane and monomethyl pentanes include hydrocarbons having at least seven carbon atoms.

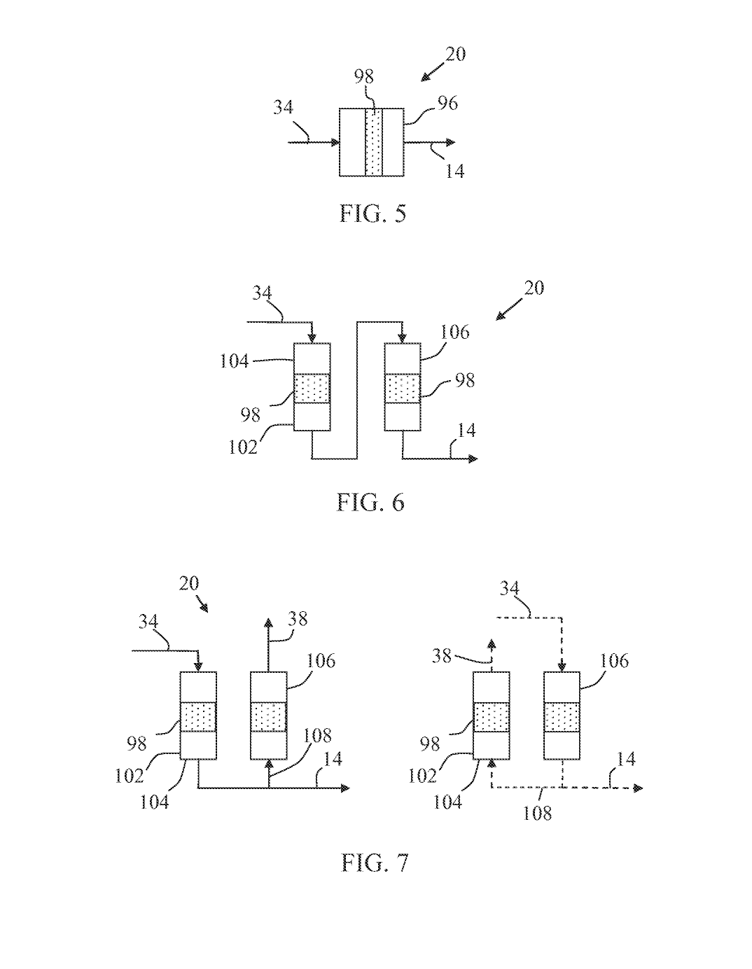

Referring now to FIG. 5, the adsorption zone 20 of FIG. 1 is illustrated in greater detail. As indicated in FIG. 1, the adsorption zone 20 receives the low aromatic hydrocarbon stream in fraction 34 from the fractionation zone 18. In FIG. 5, the adsorption zone 20 includes a single non-regenerable adsorbent bed 96 that contains an adsorbent 98 for adsorbing aromatics from the low aromatic hydrocarbon fraction 34. In an exemplary embodiment, the adsorbent 98 is a molecular sieve, such as a faujasite-type molecular sieve, and in particular a 13× or 10× molecular sieve. Typical absorbent conditions are from ambient temperature to about 60° C. (140° F.).

As the low aromatic hydrocarbon stream in fraction 34 passes through the adsorbent bed 96, aromatics are selectively adsorbed by the adsorbent 98. As a result, the aromatic-depleted product stream 14 is formed with an aromatic concentration of less than about 10 wppm, or less than about 3 wppm.

FIG. 6 illustrates another embodiment of the adsorption zone 20 of FIG. 1. In FIG. 6, the adsorption zone 20 includes two adsorption units or vessels 102 that can be arranged in series. Each adsorption unit 102 holds an adsorbent 98, such as faujasite-type molecular sieve, for example, a 13× or 10× molecular sieve. During the operation shown in FIG. 6, the low aromatic hydrocarbon stream in fraction 34 passes through the adsorption unit 102 in the lead position 104 before passing through the adsorption unit 102 in the lag position 106. The adsorbent 98 in the adsorption unit 102 in the lead position 104 is exposed to more aromatics, and adsorbs more aromatics, than the adsorbent 98 in the adsorption unit 102 in the lag position 106. Therefore, the lead position adsorbent 98 requires regeneration sooner. When regeneration is necessary for the lead adsorption unit 102, it is moved offline and the other adsorption unit 102 is moved into the lead position 104. A portion of the aromatic-depleted product stream 14 is heated and flowed through the removed adsorption unit 102. As a result, the adsorbed aromatics are desorbed into the heated portion of the aromatic-depleted product stream and removed from the adsorption unit 102 in a desorbed stream. The desorbed stream is delivered back to the processing zone 16 where the desorbed aromatics can be saturated as shown in FIG. 1.

FIG. 7 illustrates more clearly the regeneration/desorption process of the adsorption zone 20. FIG. 7 illustrates the adsorption units 102 of FIG. 6 as selectively operated in adsorption or regeneration processes. As above, each adsorption unit 102 holds an adsorbent 98, such as faujasite-type molecular sieve, for example, a 13× or 10× molecular sieve. FIG. 7 shows a first process flow (with solid flow lines) in which the fraction 34 passes through the adsorption unit 102 in the position 104 where the adsorbent 98 adsorbs aromatics therefrom to form the aromatic-depleted product stream 14. A portion 108 of the aromatic-depleted product stream 14 is heated to a temperature of about 150° C. to about 315° C. (about 300° F. to about 600° F.), such as about 260° C. (about 500° F.). The heated portion 108 is flowed through the adsorption unit 102 in the position 106. As a result, the adsorbed aromatics are desorbed into the heated portion 108 of the aromatic-depleted product stream and removed from the adsorption unit 102 in position 106 in desorbed stream 38. The desorbed stream 38 is delivered back to the processing zone 16 where the desorbed aromatics can be saturated as shown in FIG. 1.

In a second process flow (shown with dashed flow lines), the fraction 34 passes through the adsorption unit 102 in the position 106, counter to the direction of flow of the heated portion 108 in the first process flow. The adsorbent 98 adsorbs aromatics to form the aromatic-depleted product stream 14. A portion 108 of the aromatic-depleted product stream 14 is heated to a temperature of about 150° C. to about 315° C. (about 300° F. to about 600° F.), such as about 260° C. (about 500° F.). The heated portion 108 is flowed through the adsorption unit 102 in the position 104, counter to the direction of flow in the first process flow discussed above. As a result, the adsorbed aromatics are desorbed into the heated portion 108 of the aromatic-depleted product stream and removed from the adsorption unit 102 in position 104 in desorbed stream 38. The desorbed stream 38 is delivered back to the processing zone 16 where the desorbed aromatics can be saturated as shown in FIG. 1.

While specific processes and vessels are described in the embodiments of FIGS. 5 and 6, the adsorption zone 20 may conduct adsorption in the liquid phase or the vapor phase and can utilize any type of existing adsorption unit configurations such as a pressure swing, simulated moving bed, or other schemes for contacting adsorbent material with the low aromatic hydrocarbon stream in fraction 34 to remove aromatics therefrom. The operating principles of the pressure swing, thermal swing, and simulated moving bed adsorption units are known in the art.

Further, virtually any adsorbent material that has capacity for the selective adsorption of the aromatics in the low aromatic hydrocarbon stream in fraction 34 can be employed in the adsorption units. Suitable adsorbents known in the art and commercially available include crystalline material including molecular sieves, activated carbons, activated clays, silica gels, activated aluminas and the like. Typically, the adsorbents contain the crystalline material dispersed in an amorphous inorganic matrix, or binder material, having channels and cavities therein that enable liquid access to the crystalline material. A variety of synthetic and naturally occurring binder materials are available such as metal oxides, clays, silicas, aluminas, silica-aluminas, silica-zirconias, silica thorias, silica-berylias, silica-titanias, silica-aluminas-thorias, silica-alumina-zirconias, mixtures of these and the like, and clay-type binders are suitable.

### Example 1

In an example of the method for reducing an aromatic concentration in a hydrocarbon stream, the hydrocarbon stream 12 comprises paraffins, olefins, naphthenes, and benzene. In the processing zone 16, the hydrocarbon stream is combined with hydrogen 48 and passed through a saturation reactor 42. Within the saturation reactor 42, the double bonds in the aromatics are saturated with hydrogen 48 at moderate process conditions and benzene is converted to cyclohexane. The overhead stream 58 is removed from the saturated effluent 52 to form the processed stream 26 with a benzene concentration of no more than about 2 wt % benzene.

The processed stream 26 is then fractionated in the fractionation zone 18. Specifically, the processed stream 26 is delivered to the fractionation unit 80. The fractionation unit 80 separates the processed stream 26 into a bottoms fraction 32 that includes hydrocarbons having a higher boiling point than normal and cyclic hexane and monomethyl pentanes, a side draw stream 84 containing normal hexane and benzene, and overhead fraction 36 that includes branched hydrocarbons. The side draw stream 84 is then fractionated in the fractionation unit 90 to separate the low aromatic hydrocarbon fraction 34, which comprises at least about 40 wt % normal hexane.

The low aromatic hydrocarbon fraction 34 is then delivered to the adsorption zone 20. Specifically, the fraction 34 is passed through the adsorbent bed 96 where benzene is adsorbed into molecular sieves. As a result, the aromatic-depleted product stream 14 exits the adsorbent bed 96 with a benzene concentration of no more than about 10 wppm benzene and with a normal hexane concentration of at least 40 wt %.

### Example 2

In another example, a method for forming a benzene-depleted C6 product stream 14 includes fractionating a hydrocarbon stream 26 to form a C6-concentrated fraction 34 comprising C6 paraffins, C6 olefins, C6 naphthenes, and no more than about 2 wt % benzene. The hydrocarbon stream 26 is fractionated according to Example 1 and results in the C6-concentrated fraction 34. The C6-concentrated fraction 34 is introduced to the adsorption zone 20 of FIG. 6 where benzene is adsorbed from the C6-concentrated stream to form the benzene-depleted C6 product stream 14 comprising less than about 10 weight parts per million (wppm) benzene. Specifically, the C6-concentrated fraction 34 is introduced into the adsorption unit 102 in the lead position and benzene is adsorbed into the adsorbent 98 therein. A portion of the benzene-depleted C6 product stream 14 is heated and flows through the other adsorption unit 102 to desorb and remove benzene therefrom in a desorbed stream 38. A feedstock hydrocarbon stream 12 may be saturated in a saturation reactor or isomerization reactor to form the hydrocarbon stream 26.

### Example 3

In another example, the hydrocarbon stream 12 is comprised of highly pure benzene and is passed through the saturation reactor 42 with hydrogen 48. In the example, the hydrogen/benzene molar ratio is about, or more than, 3 to 1. During hydrogenation, the saturation reactor 42 is maintained at a temperature of about 290° C. and at a pressure of about 3 MPa. As a result of saturation/hydrogenation, almost all the benzene is converted into cyclohexane. The processed stream 26 exiting the processing zone 16 includes highly pure cyclohexane with no more than about 2 wt % benzene, such as no more than about 1 wt % benzene. The processed stream 26 bypasses the fractionation zone 18 and is introduced to the adsorption zone 20 where benzene is adsorbed by adsorbent 98. As a result, the aromatic-depleted product stream 14 is formed with no more than about 10 wppm benzene and substantially pure cyclohexane.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the claimed subject matter in any way. Rather, the foregoing detailed description will provide those skilled in the art with a convenient road map for implementing an exemplary embodiment or embodiments. It being understood that various changes may be made in the function and arrangement of elements described in an exemplary embodiment without departing from the scope set forth in the appended claims.

1. A method for reducing an aromatic concentration in a hydrocarbon stream comprising:
saturating aromatics in the hydrocarbon stream to form a low aromatic hydrocarbon stream comprising no more than about 2 wt % aromatics; and
passing the low aromatic hydrocarbon stream through an adsorption zone to remove aromatics therefrom to form an aromatic-depleted product stream comprising less than about 10 weight parts per million (wppm) aromatics.

2. The method of claim 1 wherein saturating aromatics in the hydrocarbon stream to form a low aromatic hydrocarbon stream comprises:
passing the hydrocarbon stream through a saturation zone and saturating double bonds in the aromatics with hydrogen to form a saturated effluent; and
fractionating the saturated effluent and recovering the low aromatic hydrocarbon stream.

3. The method of claim 2 wherein saturating aromatics in the hydrocarbon stream forms the saturated effluent comprising no more than about 1 wt % benzene.

4. The method of claim 3 wherein fractionating the saturated effluent and recovering the low aromatic hydrocarbon stream comprises recovering the low aromatic hydrocarbon stream comprising at least about 40 wt % normal hexane.

5. The method of claim 1 wherein saturating aromatics in the hydrocarbon stream to form a low aromatic hydrocarbon stream comprises:
passing the hydrocarbon stream through an isomerization zone, saturating the aromatics, and isomerizing paraffins in the hydrocarbon stream to form an isomerization effluent; and
fractionating the isomerization effluent and recovering the low aromatic hydrocarbon stream.

6. The method of claim 1 wherein the hydrocarbon stream comprises cyclohexane and benzene, and wherein saturating aromatics in the hydrocarbon stream comprises hydrogenating benzene to form cyclohexane.

7. The method of claim 1 wherein passing the low aromatic hydrocarbon stream through an adsorption zone comprises passing the low aromatic hydrocarbon stream through a single non-regenerable adsorbent vessel and adsorbing aromatics in the adsorbent vessel.

8. The method of claim 1 wherein the adsorption zone includes a first adsorbent vessel and a second adsorbent vessel, and wherein passing the low aromatic hydrocarbon stream through an adsorption zone comprises passing the low aromatic hydrocarbon stream through the first adsorbent vessel and adsorbing aromatics from the low aromatic hydrocarbon stream while regenerating adsorbent in the second adsorbent vessel.

9. The method of claim 8 further comprising:
heating a portion of the aromatic-depleted product stream; and
passing the portion of the aromatic-depleted product stream through a selected adsorbent vessel and desorbing the aromatics from the selected adsorbent vessel to regenerate the adsorbent in the selected adsorbent vessel.

10. The method of claim 9 wherein saturating aromatics in the hydrocarbon stream comprises saturating aromatics in the hydrocarbon stream in a saturation zone, wherein desorbing the aromatics from the selected adsorbent vessel into the portion of the aromatic-depleted product stream forms a desorbed stream, and wherein the method further comprises recycling the desorbed stream to saturation zone, blending with the hydrocarbon stream and saturating the aromatics therein.

11. The method of claim 1 wherein passing the low aromatic hydrocarbon stream through an adsorption zone comprises:
contacting the low aromatic hydrocarbon stream with a molecular sieve in the adsorption zone;
adsorbing aromatics into the molecular sieve; and
removing the aromatic-depleted product stream from the adsorption zone.

12. The method of claim 11 further comprising:
heating a portion of the aromatic-depleted product stream; and
contacting the portion of the aromatic-depleted product stream with the molecular sieve to desorb the aromatics therefrom.

13. The method of claim 1 wherein passing the low aromatic hydrocarbon stream through an adsorption zone comprises:
contacting the low aromatic hydrocarbon stream with a faujasite-type molecular sieve in the adsorption zone;
adsorbing aromatics into the faujasite molecular sieve; and
removing the aromatic-depleted product stream from the adsorption zone.

14. The method of claim 1 wherein:
saturating aromatics in the hydrocarbon stream forms the low aromatic hydrocarbon stream comprising no more than about 1 wt % aromatics; and
passing the low aromatic hydrocarbon stream through the adsorption zone to remove aromatics therefrom forms the aromatic-depleted product stream comprising less than about 3 wppm aromatics.

15. A method for forming a benzene-depleted C6 product stream comprising:
fractionating a hydrocarbon stream to form a C6-concentrated stream comprising C6 paraffins, C6 olefins, C6 naphthenes, and no more than about 2 wt % benzene; and
adsorbing benzene from the C6-concentrated stream to form the benzene-depleted C6 product stream comprising less than about 10 weight parts per million (wppm) benzene.

16. The method of claim 15 wherein:
fractionating the hydrocarbon stream forms the C6-concentrated stream comprising no more than about 10 wppm benzene; and
adsorbing benzene from the C6-concentrated stream forms the benzene-depleted C6 product stream comprising less than about 3 wppm benzene.

17. The method of claim 15 further comprising saturating benzene in a hydrocarbon stream to form the hydrocarbon stream comprising at least about 40 wt % normal hexane.

18. The method of claim 15 wherein adsorbing benzene from the C6-concentrated stream to form the benzene-depleted C6 product stream comprises passing the C6-concentrated stream through a single adsorbent vessel and adsorbing benzene from the C6-concentrated stream in the adsorbent vessel.

19. The method of claim 15 wherein adsorbing benzene from the C6-concentrated stream to form the benzene-depleted C6 product stream comprises passing the C6-concentrated stream through an adsorption zone, wherein the adsorption zone includes a first adsorbent vessel and a second adsorbent vessel, and wherein passing the C6-concentrated stream through an adsorption zone comprises passing the C6-concentrated stream through the first adsorbent vessel while regenerating adsorbent in the second adsorbent vessel.

20. An apparatus for reducing an aromatic concentration in a hydrocarbon stream comprising:
a saturation zone configured to receive the hydrocarbon stream and to saturate aromatics therein to form a low aromatic hydrocarbon stream comprising no more than about 2% aromatics; and
an adsorption zone configured to receive the low aromatic hydrocarbon stream from the saturation zone and to remove aromatics therefrom to form an aromatic-depleted product stream comprising less than about 10 weight parts per million (wppm) aromatics.
